(19) United States
(12) Patent Application Publication
(10) Pub. No.: US 2006/0183987 A1
(43) Pub. Date: Aug. 17, 2006

(54) Intravenous ultraviolet implant

(76) Inventor: John K. Murray, Arlington, TX (US)

Correspondence Address: John K. Murray, 2307 MONTE CARLO DR # B, ARLINGTON, TX, 76015, US

(21) Appl. No.: 11/311,856

(22) Filed: Dec. 17, 2005

Related U.S. Application Data
(60) Provisional application No. 60/638,022, filed on Dec. 20, 2004.

(51) Int. Cl. A61B 5/00

(52) U.S. Cl. 600/322

(57) ABSTRACT

Intravenous ultraviolet implant within a human body having a plurality of ultra-violet frequency (UV) LED's attached radially in an inwardly facing manner to a rigid ring, an electrical power cord attached at one end to the UV-LED ring and at the other end to a power supply such as a battery, and a housing that encloses the power supply as well as other standard electronic components such as resistors and battery contacts. A preferred embodiment includes the ring is surgically installed within a major vein so that blood passing through the the vein can be irradiated with UV light frequencies.

### CROSS REFERENCE TO RELATED APPLICATIONS

This application is based on provisional application Ser. No. 60/638,022, filed on Dec. 20, 2004.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of implantable medical devices and more specifically to an intravenous ultraviolet implant. Researchers have spent many years looking for cures for major diseases such as cancer.

The cures that have been discovered such as chemotherapy often include severe side effects and weaken the overall immune system so that other diseases may cause additional harm or death.

In addition to dangerous side effects, the current cures for cancer often do not remove all cancerous material and the cancer may return causing additional health problems including death. One method of treatment for destroying blood borne viral RNA or cancerous DNA is called phototherapy and consists of removing a patients blood and pumping it through clear tubing. As the blood travels through the tubes is is exposed an intense ultraviolet light, then pumped directly back into the patient's body. Although the process arrests cancer cell infestation of the patient's blood, it does not cure the condition. As a result, the process must be repeated as cancer cell count reaches levels detrimental to the patient's well being. The process of removing large amounts of blood from a person and replacing it is taxing on the patient's body and can not be done repeatedly over short periods of time. Therefore, there is a need for a way to expose a patient's blood to UV light in a continuous manner without having to remove the blood from the patient's body.

### BRIEF SUMMARY OF THE INVENTION

The primary object of the invention is to provide a non-pharmaceutical implant that destroys genetic molecular material that is instrumental in the process of replication of certain viral and cancerous cells that infest human blood components.

Another object of the invention is to provide a means of destroying cancerous materials within the human body without resorting to debilitating and potentially life threatening treatments such as radiation with X rays.

Another object of the invention is to provide a means of radiating a person's blood without the necessity of removing the blood from the person's body.

A further object of the invention is to provide a means of treating a patient's blood to reduce or eliminate the presence of HIV virus.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed Intravenous ultraviolet implant comprising: a plurality of ultra-violet frequency (UV) LED's attached radially in an inwardly facing manner to a rigid ring, an electrical power cord attached at one end to said UV-LED ring and at the other end to a power supply such as a battery, and a housing that encloses said power supply as well as other standard electronic components such as resistors and battery contacts. The invention is intended to be implanted in a major vein within a person afflicted with cancer or HIV aids so that the radiated light can interact with blood borne psoralens which can then interfere with the reproductive capability of cancerous or HIV cells.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a plan view of the invention.

FIG. 2 is a plan view of an incision being made in a person's vein FIG. 3 is a plan view of the invention being inserted into a person's vein.

FIG. 4 is a partial section view of the invention after being inserted into a person's vein.

FIG. 5 is a section view of a person's body showing the location of the implanted invention.

FIG. 6 as a section view of the invention.

FIG. 7 is a side view of the invention.

FIG. 8A shows a front view of an alternate design for UV LED placement

FIG. 8B shows a side view of the LED's of the alternate embodiment.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

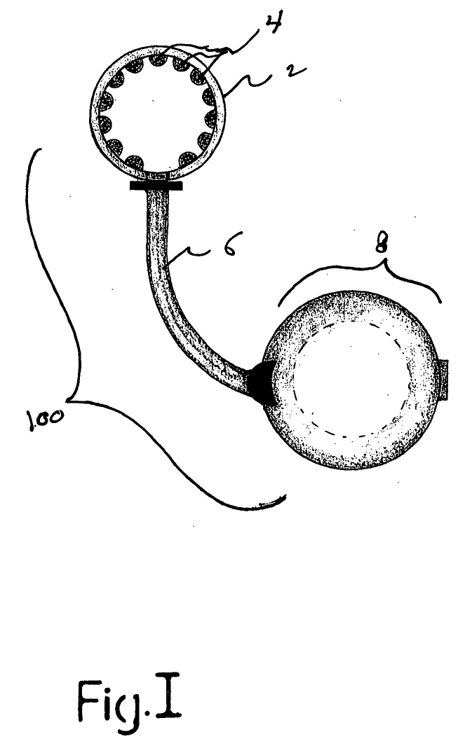

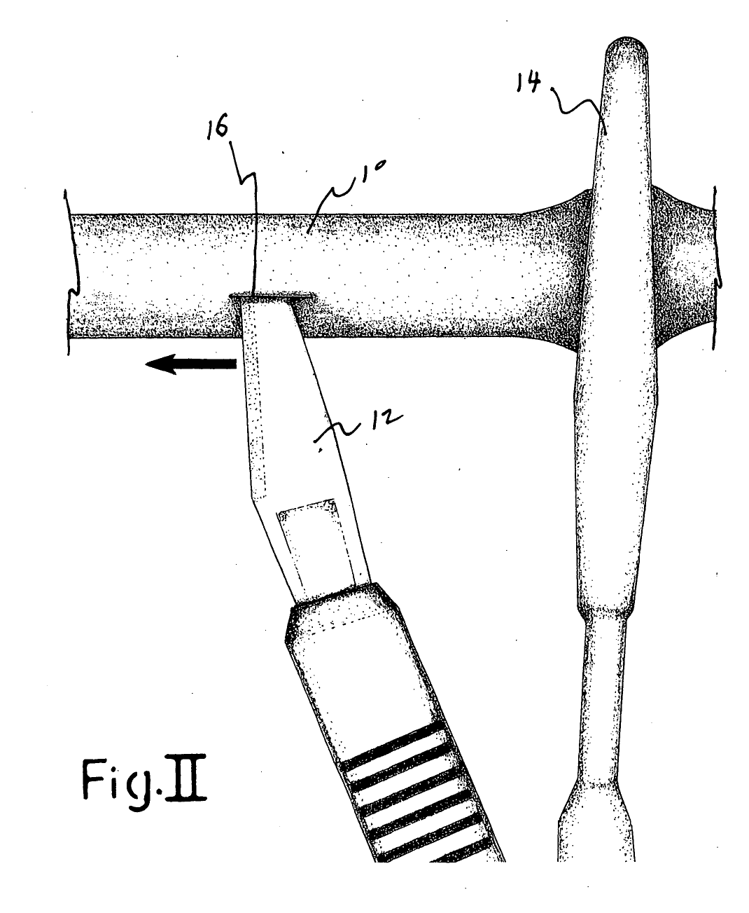

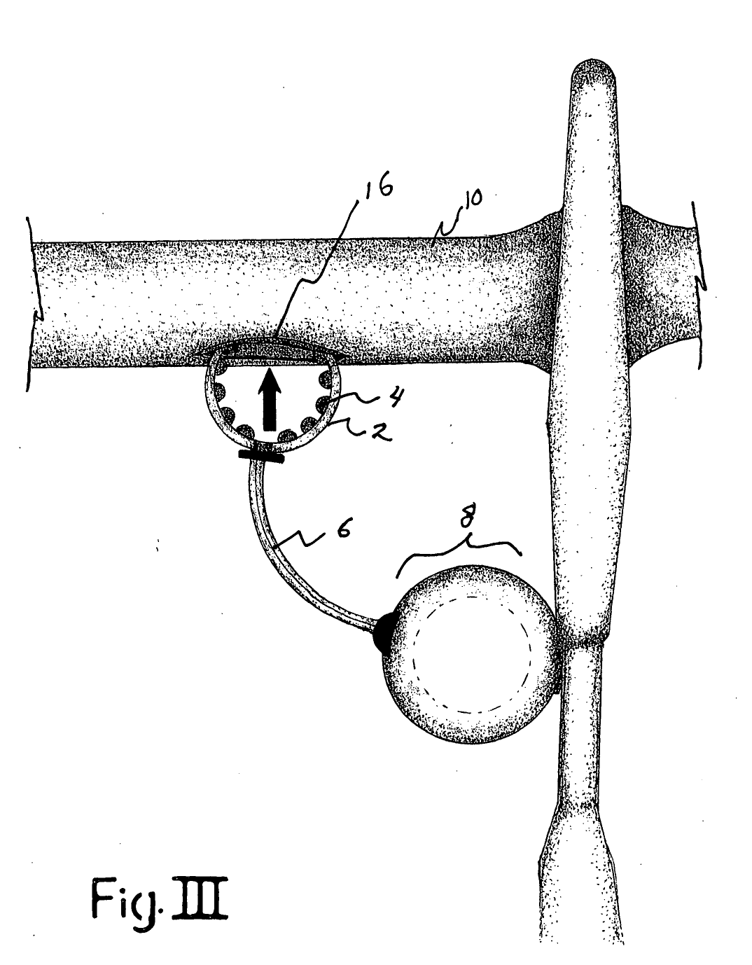

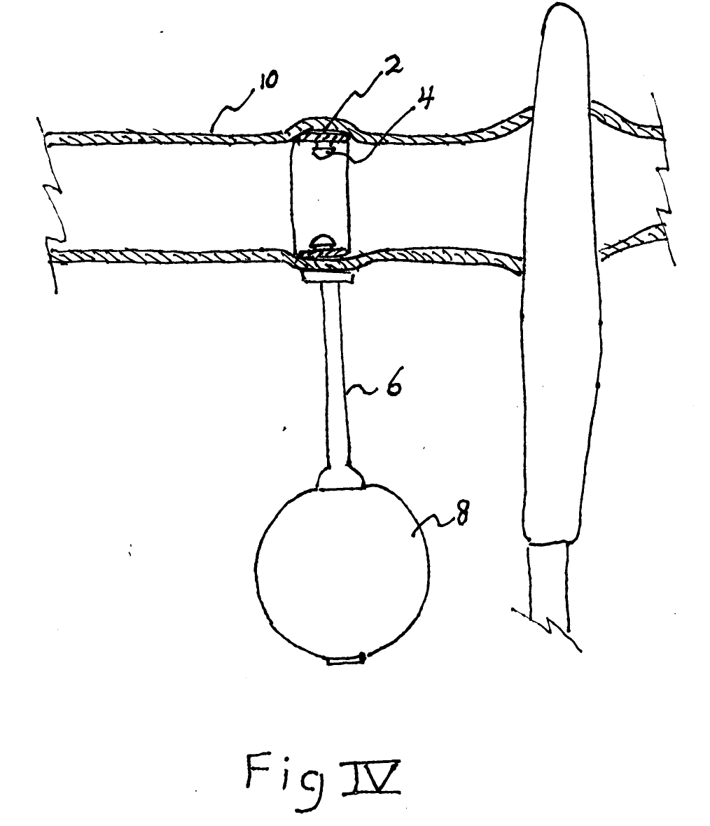

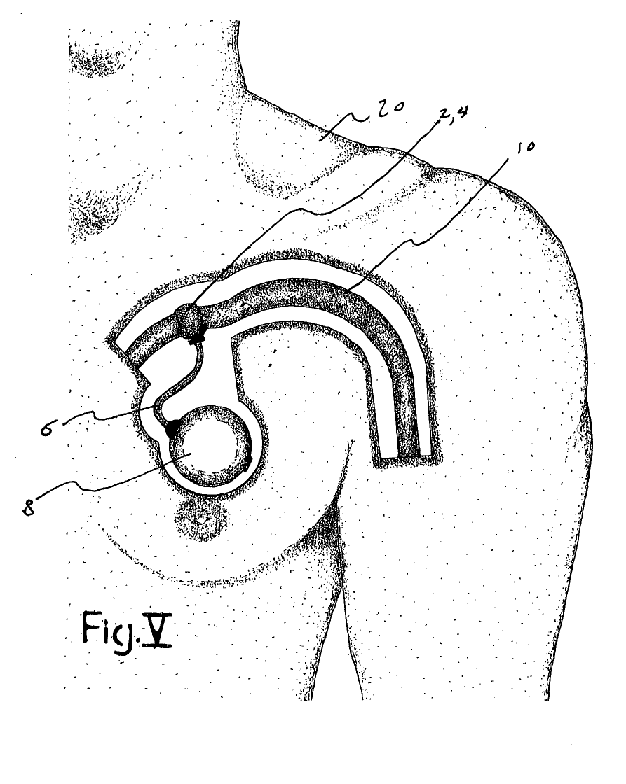

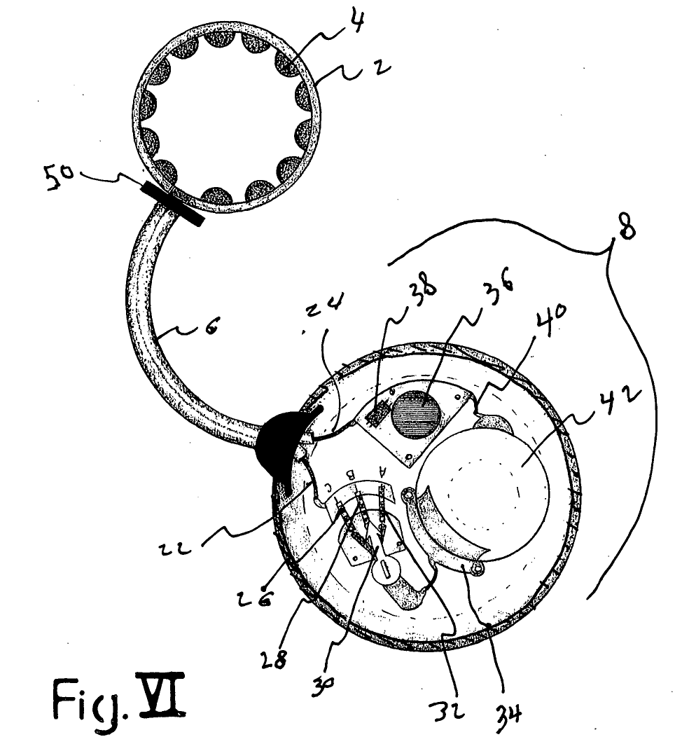

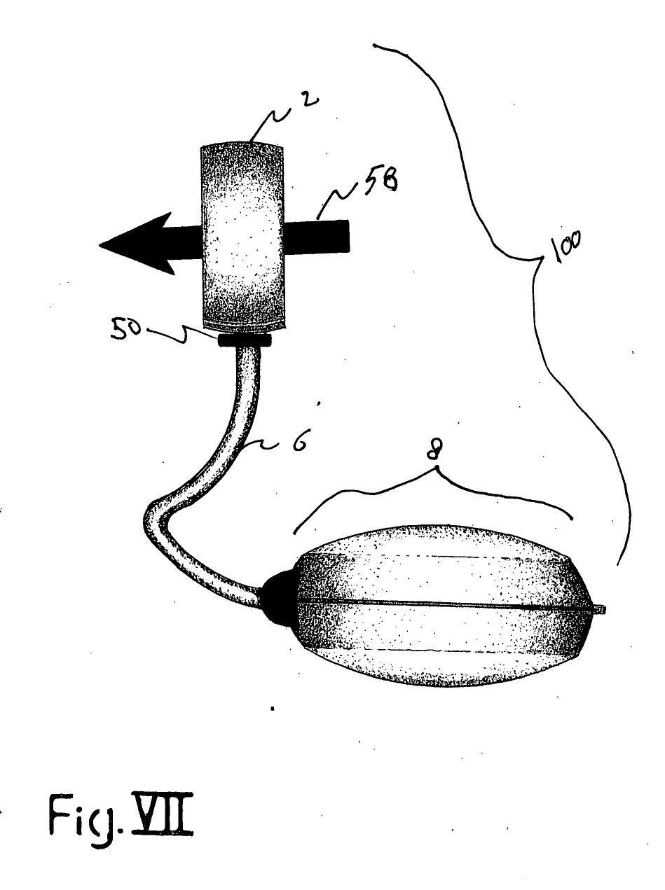

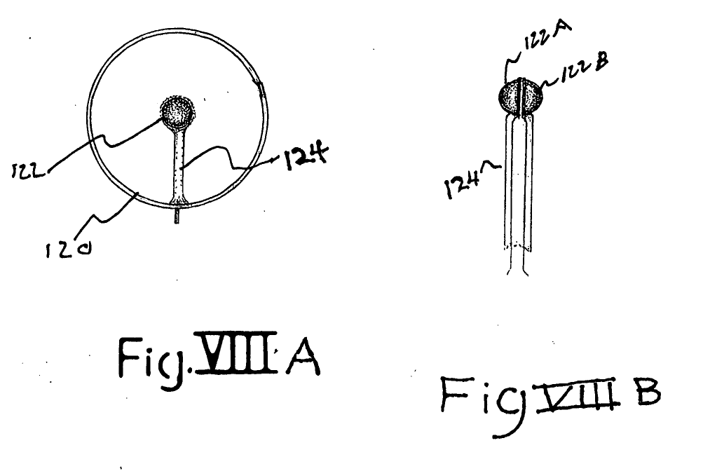

Referring now to FIG. 1 we see a plan view of the invention 100. It consists of a ring 2 that has a plurality of UV LED's 4 radially attached to the inner surface of the ring 2 so that the LED's 4 point toward the center of the ring. A resilient tubular cord 6 encloses a pair of electrically conducting wires that power the LED's 4. A Housing 8 encloses components that control and power the LED's 4 as shown in the section view on FIG. 6. The invention 100 is meant to be implanted within a vein of a person who is suffering from a disease such as cancer or HIV aids. FIG. 2 shows the beginning of an incision 16 made into the Subclavian vein 10 located in the upper-left chest cavity as shown in FIG. 5. The vein 10 is clamped 14. A second clamp (not shown) clamps the vein at the other side of the incision between the incision point and the heart. FIG. 3 shows the ring 2 portion of the invention being inserted into incision 16. After insertion, the ring is turned ninety degrees so that blood can flow through the ring 2 and be radiated by the UV LED's 4. FIG. 4 is a section view of Vein 10 and clearly shows the position of ring 2 after insertion into the vein 10. LED's 4 can be clearly seen pointing inward toward the blood that flows within the vein 10. FIG. 5 shows a preferred location for the invention within the body 20 of a person needing UV-LED treatment. FIG. 6 shows a section view of housing 8. Inside the housing are a battery 42, a UV-A, B or C select switch 30 and corresponding resistors 26, 28, 32, activation electronic signal receiver 36, 38, battery contacts 34, 40 and wire leads 24 that connect the components inside housing 8 to LED's 4. The selector switch 30 and associated resistors 26, 28, 32 can adjust the voltage going to the LED's 4 thereby adjusting the frequency of the UV light from UV-A, UV-B or UV-C. The receiving circuit 38, 36 can be used for remote activation but is not essential to the basic working of the invention. Bushing 50 is a silicone stabilizer that helps keep the ring 2 in proper alignment within the vein 10. FIG. 7 shows a side view of the invention 100 where arrow 58 indicates direction of blood flow. FIGS. 8A and 8B show an alternative embodiment of the invention where a pair of UV-LED's 122A, 122B are mounted back to back on a pedestal 124. The pedestal 124 is mounted on ring 120 so that when installed in a person's vein, blood will flow around the LED's 122A, 122B and thereby be irradiated with UV light frequencies.

When the invention is installed in a person's vein, the person is instructed to take a liquid that includes a form of psoralens called 8-methoxypsoralen (8-MOP). This substance is non-toxic and enters the blood rapidly and remains intermingled with blood components for a period of time sufficient to complete the process of exposing most of the patient's blood to intense UV light. As the psoralens in the blood pass through ring 2 and are exposed to UV light, the compound acts as a molecular surgeon, which snips away cancerous DNA within the blood, destroying its genetic programming and making it impossible for the cancerous components to replicate themselves. The fact that the entire process takes place within the patient, and not externally such as having the patients blood exit the body through a clear plastic tube, means that the process can be repeated as often as necessary without undue stress to the patient. The UV-LED lights 4 produce very little heat and therefore do not raise blood temperature. Additionally, the LED's 4 are very energy efficient, so that they can be powered for long periods of time with a relatively small battery.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Intravenous ultraviolet implant comprising:
a plurality of ultra-violet frequency (UV) LED's attached radially in an inwardly facing manner to a rigid ring;
an electrical power cord attached at one end to said UV-LED ring and at the other end to a power supply such as a battery; and
a housing that encloses said power supply as well as other standard electronic components such as resistors and battery contacts.

2. Intravenous ultraviolet implant as claimed in claim 1 wherein said ring is surgically installed within a major vein so that blood passing through the said vein can be irradiated with UV light frequencies.

3. Intravenous ultraviolet implant as claimed in claim 1 wherein said UV light can be either UV-A (between 315 and 400 nanometers) or UV-B (between 280 and 315 nanometers) or UV-C (between 100 and 280 nanometers) or any combination of said frequencies.

4. Intravenous ultraviolet implant as claimed in claim 1 wherein a person ingests psoralens (8-MOP) thereby allowing said psoralens to intermingle with said person's blood so that as said blood passes through said UV ring said UV radiation interacts with said psoralens thereby causing said psoralens to destroy cancerous DNA within said person's blood stream.
